(19) United States
(12) Patent Application Publication
(10) Pub. No.: US 2017/0181961 A1
(43) Pub. Date: Jun. 29, 2017

(54) NAIL COATING REMOVAL COMPOSITIONS

(71) Applicant: JAMBERRY NAILS LLC, American Fork, UT (US)

(72) Inventor: Douglas Dean Schoon, Dana Point, CA (US)

(21) Appl. No.: 15/164,475

(22) Filed: May 25, 2016

Related U.S. Application Data
(60) Provisional application No. 62/272,507, filed on Dec. 29, 2015.

(51) Int. Cl. A61K 8/92; A61K 8/37; A61Q 3/04; A61K 8/34

(52) U.S. Cl. CPC A61K 8/922; A61K 8/34; A61K 8/37; A61Q 3/04; A61K 2800/30

(57) ABSTRACT

A nail coating removal composition includes a solvent having an alcohol component and an ester component, and a natural oil blend. The solvent has a ratio of alcohol component to ester component of about 5:4, and the natural oil blend is added as 10-30% weight of the composition. The removal composition disrupts the adhesive bond between a solid nail coating, such as a nail wrap, strip, or film, while also disrupting the integrity of the solid nail coating itself. The removal composition enables fast nail coating removal times without damaging the nail plate or surrounding tissues.

### CROSS-REFERENCE TO RELATED APPLICATIONS

This application claims the benefit of U.S. Provisional Patent Application No. 62/272,507, filed on Dec. 29, 2015, the disclosure of which is incorporated herein in its entirety.

### BACKGROUND

The present disclosure relates generally to compositions and methods for removing decorative, cosmetic, or medical nail coatings, including coverings, films, wraps (a fabric held to the nail plate using a cyanoacrylate adhesive), polishes, adhesives, decorative self-adhesive strips (using pressure-sensitive and/or thermal-sensitive adhesive) such as shields, stickers and decals, and other products used to coat fingernails and toenails.

While beneficial, use of such nail coatings is commonly associated with damage to a wearer's nail plate. In particular, removal of such nail coatings (often involving pulling and stretching of adhesive remnants) can result in lifting and delaminating of upper layer cells of the nail plate, which causes detrimental and unwanted thinning of the nail plate.

In addition, compositions for removing nail polish from a nail or for loosening a nail coating from a nail are typically formulated with acetone or other harsh ketones as the major active ingredient. Such compositions are generally considered to provide acceptable removal of the nail covering; however, use of the compositions is also associated with drying and other damage to the nail and skin folds surrounding the nail plate. Various components included within traditional removal compositions are known to penetrate into the nail plate, contributing to drying out of the keratin in the nail plate and leading to brittle nails, a thinner nail plate, and a weaker nail edge, among other detrimental effects.

Further, typical acetone-based compositions suffer from high flammability and high volatility, and are also known to suffer from a characteristic, pungent odor that many find to be unpleasant. Although attempts have been made to develop nail polish removal compositions that reduce these undesirable effects, there exists an ongoing need for compositions that provide acceptable removal without excessive damage to the nail plate and to surrounding tissue.

In addition, typical compositions are formulated specifically for removing pigmented lacquers and enamels (most commonly referred to as “polish”), and not for the removal of nail coatings having a solid structure, such as films that are adhesively applied to the nail plate. Although some nail polish removal compositions may be used for removing a film, the long exposure times required exacerbate the detrimental effects to the nail and surrounding tissues. In addition, wearers of such nail coatings may become impatient with the length of the required exposure times, and may prematurely force the coating off of the nail, leading to further damage to the nail plate and particularly to the edge of the nail plate.

### BRIEF SUMMARY

Certain embodiments described herein relate to a nail coating removal composition comprising a solvent having an alcohol component and a short chain ester component and a natural oil blend. In some embodiments, the alcohol component is a short-chain alcohol such as isopropyl alcohol, and the short chain ester component is an alkyl ester of an organic acid, such as methyl acetate.

It has been found that nail coating removal compositions of the present disclosure are able to beneficially protect the nail plate, aid in the regeneration of damaged nail plates and other nail tissue, and provide the nail plate with greater resistance to future damage. In addition, when a nail coating removal composition is formulated according to one or more embodiments disclosed herein, the removal composition can provide such benefits to nail tissues while also functioning to effectively remove the nail coating.

In some embodiments, the ratio of the alcohol component to the short chain ester component is about 1:4, 1:3, 1:2, 2:3, 4:5, 1:1, 55:45, 5:4, 3:2, 3:1, or 4:1. In some embodiments, the alcohol component makes up about 10-90% by weight, or about 30-80% by weight, or about 40 to 70% by weight, or about 45 to 60% by weight of the solvent.

In some embodiments, the natural oil blend includes one or more C8 to C18 free fatty acids, C8 to C18 fatty acid alcohols, and/or plant-based natural oils such as cottonseed (e.g., winterized type), flaxseed, grapeseed, coconut, avocado, argan, babussu, wheat germ, hempseed, corn, safflower, poppyseed, sunflower, walnut, soya bean, canola, chia seed, perilla seed, palm kernel, peanut, palm, jojoba, macadamia nut, sesame seed, meadowfoam seed, pumpkin seed, and olive oil; caprylic, capric, lauric, myristic, palmitic, stearic, palmitoleic, oleic, linoleic, linolenic, and stearidonic acid, and related fatty acid alcohols.

In some embodiments, the natural oil blend is included in amount ranging from about 5 to 90%, or about 5 to 50%, or about 10 to 30%, or about 10 to 20%, or about 10 to 15% of the weight of the composition. In some embodiments, the natural oil blend has a high concentration of C12 to C18 fatty acids and/or fatty acid alcohols. In preferred embodiments, the natural oil blend has a high concentration of C16 to C18 fatty acids and/or fatty acid alcohols, and more particularly a high concentration of C18 fatty acids and/or fatty acid alcohols. In one example, the natural oil blend may be formulated with a fatty acid profile of about 90% or more, or about 95% or more, or about 97% or more of C12 to C18 fatty acids and/or fatty acid alcohols. In another example, the natural oil blend is formulated with a fatty acid profile of about 90% or more, or about 93% or more, or about 95% or more, or about 97% or more of C16 to C18 fatty acids and/or fatty acid alcohols. In another example, the natural oil blend is formulated with a fatty acid profile of about 60-95%, or about 65-90%, or about 70-85% of C18 fatty acids and/or fatty acid alcohols. In some embodiments, the natural oil blend has a C18 fatty acid profile of greater than about 65%, 70%, 75%, 80%, 85%, 90%, or 95%.

In some embodiments, the amount of fatty acid alkyl esters is less than 25%, less than 10%, less than 5%, or less than 1% by weight of the composition. In some embodiments, fatty acid alkyl esters not already included naturally within the natural oil blend are omitted. In some embodiments, substances (such as urea) that are insoluble in the removal composition at room temperature are omitted.

In some embodiments, a method of removing a nail coating from the nail plate of a wearer includes: (1) providing a nail coating removal composition as described herein; (2) contacting the nail coating removal composition to a nail coating; and (3) removing the nail coating from the nail plate. In some embodiments, a method of removing a nail coating from a nail plate further includes, after contacting the nail coating removal composition to the nail coating, allowing the nail coating removal composition to disrupt at least one of the cohesive integrity of the nail coating and/or the adhesive bond between the nail coating and the nail plate. In some embodiments, the nail coating is a solid nail coating, such as a nail strip, shield, wrap or film, and the nail coating removal composition enables removal by disrupting the cohesive integrity of the nail coating itself and/or by disrupting the adhesive bond between the nail coating and the nail plate (e.g., through wicking action of one or more of the natural oils included in the composition).

### DETAILED DESCRIPTION

### Definitions

As used herein, the terms “nail,” “nail plate,” “cuticle,” “nail folds,” “nail bed,” “nail edge,” and other anatomical terms referring to the nail and surrounding tissues refer to fingernails and toenails. Accordingly, description having a specific reference to a fingernail in a particular embodiment should be understood to apply, in other embodiments, to a toenail as well, and vice versa.

As used herein, the terms “nail coating,” “nail cover,” “nail covering,” and similar terms refer to an article that is coated, positioned, or layered onto a nail plate for one or more cosmetic, decorative, medical, or other aesthetic and/or functional purposes. The term “nail coating” and similar terms include liquid compositions that cure after application to the nail plate, such as common nail polishes. Such compositions may be referred to herein as “liquid nail coatings.” It will be understood, however, that these compositions are only applied as liquids, and afterwards take on a solid form upon curing on the nail plate.

The term “nail coating” and similar terms also refer to a solid piece of material (e.g., a strip, shield, sticker, decal or film made from a vinyl polymer or from other materials) configured to be adhered to the nail plate to cover at least a portion of the nail plate. Such articles may be referred to herein as “solid nail coatings.” Although various terms are used in the art to refer to such solid nail coatings, one of skill in the art will understand that many solid nail coverings include an adhesive layer or are applied to a nail plate using an applied adhesive that allows the solid nail coating to be sealed to the nail plate using pressure or a combination of pressure and elevated temperatures. For example, a thermal-setting adhesive can be formulated to set and/or set more securely with the application of heat.

In one example, an adhesive suitable for use with a solid nail covering is an acrylate polymer, such as an acrylate(s)/ethylhexyl acrylate copolymer. Embodiments of removal compositions described herein have been shown to be particularly effective in removing thermal-setting and/or pressure-setting solid nail coverings applied using such adhesives. Exemplary solid nail coatings usable with the removal compositions disclosed herein include those nail coatings (including coverings, wraps, stickers, etc.) sold under the trade names of Jamberry® or Jamberry Nails®.

As used herein, the terms “removal time,” “removal rate,” and similar terms refer to the duration of time for a wearer of a nail coating to expose the nail coating to the nail coating removal composition and wipe, peel, lift, or otherwise remove the nail coating completely from the nail plate. In situations where the nail coating is a solid nail coating, such as a decorative adhesive nail film or strip, the removal time represents the amount of time taken for a user to contact the nail coating and nail with the nail coating removal composition and to peel, lift, or slide the nail coating from the nail plate without resistance and without damaging the nail plate or surrounding tissues.

As used herein, the term “natural oil” and similar terms refer to various plant-based oils including, but not limited to, cottonseed, flaxseed, grapeseed, coconut (typically labeled as 76°, 91°, 92°, etc., referring to the melting point of the oil in degrees F., and also commonly available in fractionated form with a higher proportion of C8 and C10 fatty acids), avocado, argan, babussu, hempseed, corn, safflower, poppyseed, sunflower, walnut, soya bean, canola, chia seed, perilla seed, palm kernel (flakes or oil), peanut, palm, jojoba, macadamia nut, sesame seed, meadowfoam seed, pumpkin seed, wheat germ, and olive oils. Other plant-based oils suitable for safe application to the nail plate and surrounding tissues are also included within this disclosure.

The term “natural oil” and similar terms also include extracted or purified saturated and unsaturated fatty acids, particularly fatty acids within the C8 to C18 range, such as caprylic acid (C8), capric acid (C10), lauric acid (C12), myristic acid (C14), palmitic acid (C16), stearic acid (C18), palmitoleic acid (C16:1), oleic acid (C18:1), linoleic acid (C18:2), linolenic acid (C18:3), and stearidonic acid (C18:4), for example.

As used herein, the term “natural oil” and similar terms also include alcohols of fatty acids (e.g., a natural reduction product of one or more fatty acids), such as capryl alcohol (C8), capric alcohol (C10), lauryl alcohol (C12), myristyl alcohol (C14), cetyl alcohol (C16), stearyl alcohol (C18), and palmitoleyl alcohol (C16:1), for example.

The term “natural oil blend,” “oil component,” and similar terms refer to an amount of a natural oil, fatty acid, fatty acid salt, or fatty alcohol in pure form or in combination with one or more additional natural oils and/or other additives.

The terms “additives,” “additional ingredients,” “added components,” and the like can include antioxidants (e.g., Tocopherol) or other preservatives, skin or nail conditioning agents, surfactants, carrier agents, mono-, di-, or tri-glycerides, glycerol, colorants, fragrances, and/or other beneficial ingredients. Alkyl esters of fatty acids may also be included; however, in preferred embodiments, the amount of fatty acid alkyl esters is less than 50%, less than 25%, less than 10%, less than 5%, or less than 1% by weight of the natural oil blend or of the overall composition. For example, preferred embodiments include only those fatty acid alkyl esters that occur naturally in the oils of the composition without chemical modification.

Unless specifically described otherwise, all percentages and other ratios are expressed as % wt. or as ratios based on relative weights of the components or sub-components of the nail coating removal compositions.

The terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition. In addition, unless expressly described otherwise, all stated amounts (e.g., concentrations, percentages, ratios, etc.) are to be interpreted as being “approximately,” “about,” and/or “substantially” the stated amount, regardless of whether the terms “approximately,” “about,” and/or “substantially” are expressly stated in relation to the stated amount(s).

Nail Coating Removal Compositions

In some embodiments, a nail coating removal composition includes: (1) a solvent comprised of (i) an alcohol component, and (ii) a short chain ester component; and (2) a natural oil blend. In preferred embodiments, the alcohol component includes a short-chain alcohol (having an alkyl chain of 1 to 3 carbon atoms), and most preferably, the alcohol component includes isopropyl alcohol (IPA).

In some embodiments, the alcohol component is a solution of at least about 50% alcohol, or at least about 70% alcohol. Preferably, the alcohol component includes at least 75% alcohol, at least 80% alcohol, at least 85% alcohol, at least 90% alcohol, at least 95% alcohol, or at least 99% alcohol. In preferred embodiments, the alcohol component is a 99% IPA solution (having approximately 99% IPA and approximately 1% water). In other embodiments, the alcohol component may include a lower concentration of IPA (e.g., about 50%, 70%, 91%, 95%, or values between about 70% and 95%).

It has been found that embodiments having an alcohol component with a higher concentration of IPA exhibit greater solubility of the natural oil blend and other added components, thereby providing a composition with greater consistency and reduced or eliminated tendency for components to crystalize or fall out of solution and reduce the effectiveness and/or appearance of the composition. For example, many free fatty acids and/or oils can crystalize or form precipitates if the concentration is too high or the temperature becomes too low.

Similarly, nail coating removal compositions preferably have reduced levels of, or omit, substances that are insoluble or that tend to fall out of solution (e.g., at temperatures of about room temperature). For example, in some embodiments, substances that are insoluble or have low solubility in high concentrations of IPA (e.g., urea and urea-based compounds) are omitted in order to allow the inclusion of IPA at the beneficial high concentrations without the detrimental effects (e.g., to appearance and/or consistency) of the insoluble substances. For example, urea has been found to be insoluble in 99% IPA and in natural oils, and has limited solubility in other common grades of IPA (e.g., 91% IPA and 70% IPA).

The short chain ester component is preferably an alkyl ester of an organic acid, such as an alkyl ester of a carboxylic acid. In preferred embodiments, the ester component is a volatile solvent, such as an alkyl ester of acetic acid, e.g., methyl acetate or ethyl acetate. Methyl acetate is more preferred, as it has lower volatility relative to ethyl acetate (and is not considered a volatile organic compound (VOC) under some regulations that limit VOC content), and has also been found to provide synergistic benefits in the effectiveness of nail coating removal compositions, as explained in greater detail below.

The natural oil blend includes one or more natural oils selected to enhance the consistency, appearance, and/or tissue effects (e.g., tissue protecting, tissue conditioning, and/or lipid regenerating properties) of the nail coating removal composition. In some embodiments, the natural oil blend is provided in an amount ranging from about 5 to 50%, or about 10 to 30%, or more preferably about 10 to 20%, or 10 to 15% by weight of the composition.

In other embodiments, the natural oil blend is included in an amount that is higher than 50%. For example, when a natural oil blend has a lower relative melting temperature, the natural oil blend may be included in an amount of about 50 to 90% by weight of the composition. In one example, compositions having high levels of cottonseed, hempseed, and/or wheat germ oils may have a natural oil blend included in an amount of about 50 to 90% by weight of the composition.

In some embodiments, the natural oil blend has a high concentration of C12 to C18 fatty acids and/or fatty acid alcohols. In preferred embodiments, the natural oil blend has a high concentration of C16 to C18 fatty acids and/or fatty acid alcohols, and more particularly a high concentration of C18 fatty acids and/or fatty acid alcohols. In one example, the natural oil blend may be formulated with a fatty acid profile of about 90% or more, or about 95% or more, or about 97% or more of C12 to C18 fatty acids and/or fatty acid alcohols. In another example, the natural oil blend is formulated with a fatty acid profile of about 90% or more, or about 93% or more, or about 95% or more, or about 97% or more of C16 to C18 fatty acids and/or fatty acid alcohols. In another example, the natural oil blend is formulated with a fatty acid profile of about 60-95%, or about 65-90%, or about 70-85% of C18 fatty acids and/or fatty acid alcohols. In some embodiments, the natural oil blend has a C18 fatty acid profile of greater than about 65%, 70%, 75%, 80%, 85%, 90%, or 95%.

As explained in greater detail below, it has been found that the natural oil blend, included within the disclosed ranges, enables enhanced removal of nail coatings with reduced or eliminated detrimental effects to the nail plate. For example, lipid and free fatty acid regeneration can prevent cracking of the skin surrounding the nail plate, and the condition of the nail plate of a user can thereby improve with each use of a removal composition. Testing has shown that users see improvements to flexibility and toughness/durability of the nail plate when the removal compositions described herein are utilized. In some embodiments, the combination of solvent and natural oil blend function to synergistically reduce nail coating removal times.

In preferred embodiments, the natural oil blend is formed of one or more of cottonseed oil, flaxseed oil, grapeseed oil, hempseed oil, chia seed oil, pumpkinseed oil, perilla seed oil, and/or wheat germ oil. Some embodiments include one or more natural oils mixed with one or more of a lauric acid, caprylic acid, myristic acid, stearic acid, oleic acid, linoleic acid, myristic alcohol, cetyl alcohol, and/or stearyl alcohol component, which may be added to make up about 0.5 to 40%, or about 1 to 30%, or about 2 to 20% of the composition of the natural oil blend component. These oils/components, and blends including one or more of these oils/components, have been found to particularly enhance the removal rate of a nail coating removal composition and/or particularly enhance the nail protecting and regenerating properties when included within the composition.

In preferred embodiments, the solvent is formulated to have an alcohol component making up about 10-90% by weight, or about 30-80% by weight, more preferably about 40 to 70% by weight, and even more preferably about 45 to 60% by weight of the solvent. For example, a solvent having an alcohol component formed as IPA and an ester component formed as methyl acetate may have a ratio of IPA to methyl acetate of about 1:4, 1:3, 1:2, 2:3, 4:5, 1:1, 5:4, 3:2, 3:1, or 4:1, with ratios of about 2:3, 4:5, 1:1, 5:4, 3:2, and 3:1 being more preferred, and ratios of about 4:5, 1:1, 55:45, or 5:4 being even more preferred.

### Mode of Action

Suprisingly, it has been found that the combination of a solvent formed from an alcohol and an alkyl ester of an organic acid with a natural oil blend is able to decrease the removal time of nail coatings while simultaneously reducing or eliminating the detrimental effects associated with removal of nail coatings and/or removal using traditional nail coating removal compositions (e.g., drying of nail plate and surrounding tissues, peeling and cracking of edge of nail plate, etc.)

Without being bound to any particular theory, it is thought that one or more components of the composition function to weaken or degrade the nail coating (e.g., by disrupting the internal cohesion of the covering), while one or more separate components of the composition function to weaken or degrade the interface between the nail plate and the nail coating (e.g., by disrupting the external adhesion of the coating to the nail plate, such as by disrupting the adhesive attaching the coating to the nail plate). This dual mode of action beneficially enhances the removal rate of the composition, leading to more effective, faster, and/or less-damaging removal and greater rates of user compliance with proper removal techniques. In addition, in at least some embodiments the dual mode of action is tailored to provide effective removal of nail coatings without excessive disruption of cohesion. For example, at least some embodiments are formulated to provide enough distortion of the coating to speed removal without causing the coating to break apart before the interfacial bond between the coating and the nail plate is sufficiently disrupted.

For example, in applications directed to the removal of a solid nail coating, the adhesive used to bind the solid coating to the nail plate has been found to exhibit a degree of structural porosity. The nail removal composition, and particularly the alcohol component of the solvent and the natural oil blend, has been found to be capable of wicking into the internal matrix of the binding adhesive (e.g., through capillary action actuated by the porous structure of the adhesive matrix). By wicking or otherwise migrating into the interstitial space between the nail coating and the nail plate, these components function to bring the removal composition into contact with the adhesive to disrupt the adhesion between the nail coating and the nail plate.

Beneficially, this wicking function more rapidly brings the removal composition into contact with the adhesive at all areas along the interface between the nail coating and nail plate, as opposed to merely contacting the adhesive at or near the edges of the interface or only moving inwards as the nail coating is progressively removed to expose a greater portion of the nail plate. This action can lead to faster removal of the nail coating.

In some embodiments, the natural oil blend also functions to disrupt the adhesive by wicking into the interstitial matrix of the adhesive. In some embodiments, the natural oil blend has been found to enhance distribution of the removal composition throughout the matrix of the adhesive, such that the alcohol component and the natural oil blend are capable of synergistically functioning together to provide faster contact with and disruption of the adhesive.

In preferred embodiments, the nail coating removal composition omits additional fatty acid alkyl esters not included naturally within the oils. For example, in applications for removal of a solid nail coating, a natural oil blend component having a greater proportion of non-esterified fatty acids (i.e., fatty acids that maintain the carboxylic acid group) to fatty acid esters has been found to provide greater wicking of the composition into the matrix of the adhesive at the interface between the nail plate and the nail coating.

In some embodiments, a removal composition, and particularly the short chain ester component of the solvent of the removal composition, is capable of weakening or degrading the internal cohesion of the nail coating. For example, in applications where the nail coating is a solid nail coating, the short chain ester component of the solvent has been found to weaken the integrity of the solid nail coating. In some circumstances, this enables easier removal of the solid nail coating from the nail plate (e.g., by allowing a user to more easily bend, fold, or otherwise manipulate the covering as it is peeled or lifted off the nail plate).

As described above, the alcohol component is preferably included in a range of about 30 to 80%, or about 40 to 70%, or more preferably about 45 to 60% by weight of the solvent. In addition, the short chain ester component is preferably included in a range of about 20 to 65%, or about 30 to 55%, or more preferably about 40 to 50% by weight of the solvent. For example, use of methyl acetate at levels above about 50% has been found in some circumstances to cause a solid nail coating to which it is applied to break or disintegrate into pieces. In some situations, this may be undesirable as it may require a user to remove the covering piece by piece rather than as one integral whole. Alternatively, in some situations it may be desirable to degrade the nail coating into separate pieces, such as by including the ester component of the solvent in an amount higher than about 50%.

In preferred embodiments, insoluble constituents and constituents with a tendency to crystalize or fall out of solution are omitted or reduced to levels at which their effects are negligible. The reduction or omission of such substances is beneficial for a number of reasons. For example, avoiding the formation of crystalized products and/or solid precipitates avoids potential hampering of the advantageous wicking effect described above, e.g., as a result of precipitate blocking passage of the compositor through the adhesive matrix or otherwise adjusting the fluid dynamics of the removal process. Accordingly, preferred embodiments omit urea and other compounds known to have low solubility in solutions having a high concentration of IPA.

### Methods of Use

In some embodiments, a method of removing a nail coating from a nail plate of a wearer includes: (1) providing a nail coating removal composition, the nail coating removal composition including (i) a solvent comprised of an alcohol component and a short chain ester component, and (ii) a natural oil blend; (2) contacting the nail coating removal composition to a nail coating; and (3) removing the nail coating from the nail plate.

In some embodiments, a method of removing a nail coating from a nail plate further includes, after contacting the nail coating removal composition to the nail coating, allowing the nail coating removal composition to disrupt at least one of the cohesive integrity of the nail coating and the adhesive bond between the nail coating and the nail plate. In some embodiments, the nail coating is a solid nail coating, such as a nail strip or film, and the nail coating removal composition enables removal by disrupting the cohesive integrity of the nail coating (e.g., through action of the short chain ester component of the solvent) and by disrupting the adhesive bond between the nail coating and the nail plate (e.g., through action of the alcohol component of the solvent and/or the natural oil blend).

The nail coating removal methods described herein beneficially enable effective removal without exacerbating detrimental effects to the nail plate and/or surrounding tissues. In addition, the relatively rapid removal rates can increase user compliance with proper removal protocol, as opposed to causing impatience and resulting in premature removal with associated nail plate damage.

### EXAMPLES

### Example 1

Solid nail coatings with adhesive layers formed from an acrylate(s)/ethylhexyl acrylate copolymer were each affixed (by pressure-sealing and heat-sealing) lengthwise to a respective 0.75 inch diameter round glass tubes. Before adhering each nail coating to its respective tube, a thin nylon wire was passed transversely across and under the width of the coating. The nylon wire was then passed out of the center of the nail coating so that the tube could be suspended (and balanced evenly) by the nylon wire. The tubes were filled with steel beads to a weight of 100 grams +/−0.01 grams. Each tube was then submerged into a bath containing a nail coating removal composition, and a timer measured the length of time for the tube to break free and drop to the bottom of the bath. Times ranged from between 1 to 6 minutes. Compositions including coconut, grapeseed, cottonseed, and/or flaxseed gave the best results. An IPA to methyl acetate ratio of 55:45, and an oil content of about 10% by weight, provided the best results.

### Example 2

Nail removal compositions were tested on solid nail coatings (having acrylate(s)/ethylhexyl acrylate copolymer adhesive layers) attached to several wearer's nails by pressure and heat sealing. Typically, after 3 to 7 days of wear, users dipped a cotton pad into the remover composition, and then rubbed the wetted pad over the top of the nail coating for a time sufficient to wet the entire nail coating. Then using the saturated pad and a fingernail, the wearer caught a strip edge that had begun to loosen and then gently worked the exposed underside backward with the wetted pad and their finger, progressively exposing more and more of the underside of the solid coating material. Compositions including coconut, grapeseed, cottonseed, and/or flaxseed gave superior results. An IPA to methyl acetate ratio of about 55:45, and an oil content of about 10% by weight, gave superior results. In some instances, safe removal times of 5 to 10 seconds per solid nail coating were achieved.

### Example 3

Five users between the ages of 20 and 45 were asked to test various removal compositions by using the compositions to remove solid nail coatings (adhered to the nail plates using an acrylate(s)/ethylhexyl acrylate copolymer adhesive layer) according to the procedure described in Example 2 but with removal being initiated after 3 to 5 days of wear. Each removal composition tested in this example was used to remove four solid nail coverings per user. Removal times for each solid nail covering were measured and recorded. The removal compositions included a solvent having varying concentrations of 99% IPA with the remainder of the solvent made up of methyl acetate. Each removal composition also included cottonseed oil at a 10:1 ratio of cottonseed oil to solvent. Results are shown in Table 1.

TABLE 1

| % wt. IPA (99%) | Average removal time |
|---|---|
| of solvent | (seconds) |
| 0 | 32 |
| 10 | 31 |
| 20 | 30 |
| 30 | 23 |
| 40 | 24 |
| 45 | 19 |
| 50 | 21 |
| 60 | 18 |
| 70 | 40 |
| 80 | 32 |

### Example 4

Various formulations including urea were tested and removal times were recorded following the removal procedure described in Example 2, with removal being initiated after 3-5 days of wear. Five users were tested, and each separate formulation was used on 6-10 different fingers (no more than two fingers on the same user). The solid nail coatings were adhered by heat-sealing and pressure-sealing an acrylates/ethylhexyl acrylate copolymer adhesive layer to the respective nail plate). The tested compositions each included a solvent base of 91% IPA and methyl acetate at a 55:45 ratio by weight of the composition, with varying amounts of urea and lauric acid added. Results are shown in Table 2.

TABLE 2

| | | Average Removal Time |
|---|---|---|
| % wt. lauric acid | % wt. urea | (seconds) |
| 0 | 0 | 33 |
| 40 | 0 | 19 |
| 0 | 7 | 28 |
| 20 | 0 | 20 |
| 0 | 3.5 | 25 |
| 20 | 3.5 | 26 |

As shown in Table 2, compositions including lauric acid and omitting urea performed better than those having urea without lauric acid or those having urea and lauric acid. In the composition including both lauric acid and urea, it is likely that the urea formed chemical complexes with the lauric acid (urea being known to complex with fatty acids; see, e.g., Progress in the chemistry of fats and other lipids, Vol. 2, Pergamon Press, NY, 1954, pp. 243-267; Swern et al., J. Am. Oil Chem. Soc. 1952, Vol. 29:10, pp. 431-434), neutralizing the effectiveness of both components as a removal accelerator.

### Examples 5-19

In Examples 5-19, various different formulations were tested, and corresponding removal times were recorded, following the removal procedure described in Example 2, with removal being initiated after 3-5 days of wear. Unless otherwise noted, for each Example, 3-5 users tested the formulations. The solid nail coatings were adhered by heat-sealing and pressure-sealing an acrylate(s)/ethylhexyl acrylate copolymer adhesive layer to the respective nail plate. The tested compositions each included a solvent base of 99% IPA and methyl acetate at a 55:45 ratio by weight of the composition.

### Example 5

Various formulations having different levels of lauric acid (some also having 10% wt. cottonseed oil) were tested. Results are shown in Table 3. Formulations appeared to work best with lauric acid included in the 20-40% range. Additional testing has shown that lauric acid formulations also work well at concentrations lower than 20%. At concentrations above 7%, solutions tended to exhibit solidification and crystallization.

TABLE 3

| | | Average Removal Time |
|---|---|---|
| % wt. Lauric Acid | % wt. Cottonseed Oil | (seconds) |
| 0 | 0 | 31 |
| 10 | 0 | 26 |
| 20 | 0 | 24 |
| 30 | 0 | 20 |
| 40 | 0 | 23 |
| 0 | 10 | 33 |
| 10 | 10 | 25 |
| 20 | 10 | 30 |
| 30 | 10 | 25 |
| 40 | 10 | 22 |

### Example 6

Various formulations having different levels of a capric and caprylic acid blend (at about 1:1) were tested, along with a formulation of the capric and caprylic acid blend mixed with lauric acid (at 20% wt. capric/caprylic acid blend and 20% wt. lauric acid). Results are shown in Table 4. Formulations appeared to work well at 30-40% wt. capric/caprylic acid blend. The capric/caprylic acid blend also appeared to work synergistically with lauric acid, and best results were seen with the capric/caprylic acid blend and lauric acid combination.

TABLE 4

| % wt. Capric/Caprylic | | Average removal time |
|---|---|---|
| Acid Blend | % wt. Lauric Acid | (seconds) |
| 0 | 0 | 29 |
| 10 | 0 | 23 |
| 20 | 0 | 23 |
| 30 | 0 | 20 |
| 40 | 0 | 20 |
| 20 | 20 | 18 |

### Example 7

Various formulations having different levels of myristic acid were tested, along with a formulation having myristic acid mixed with lauric acid (at 20% wt. each). Results are shown in Table 5. The myristic acid appeared to work synergistically with lauric acid, and best results were seen with the myristic acid and lauric acid combination.

TABLE 5

| | | Average removal time |
|---|---|---|
| % wt. Myristic Acid | % wt. Lauric Acid | (seconds) |
| 0 | 0 | 22 |
| 10 | 0 | 16 |
| 20 | 0 | 15 |
| 30 | 0 | 19 |
| 20 | 20 | 12 |

### Example 8

Various formulations having different levels of oleic acid were tested, along with a formulation having oleic acid mixed with lauric acid (at 30% wt. each). Results are shown in Table 6.

TABLE 6

| | | Average removal time |
|---|---|---|
| % wt. Oleic Acid | % wt. Lauric Acid | (seconds) |
| 0 | 0 | 25 |
| 10 | 0 | 18 |
| 20 | 0 | 24 |
| 30 | 0 | 20 |
| 40 | 0 | 23 |
| 30 | 30 | 31 |

### Example 9

Various formulations having different levels of palmitic acid were tested, along with a formulation having palmitic acid mixed with lauric acid (at 10% wt. each). Results are shown in Table 7.

TABLE 7

| | | Average removal time |
|---|---|---|
| % wt. Palmitic Acid | % wt. Lauric Acid | (seconds) |
| 0 | 0 | 33 |
| 10 | 0 | 22 |
| 20 | 0 | 51 |
| 10 | 10 | 24 |

### Example 10

Various formulations having different levels of stearic acid were tested, along with a formulation having stearic acid mixed with lauric acid (at 10% wt. each). Results are shown in Table 8. Notably, stearic acid was found to crystallize at concentrations higher than about 10%, slowing removal. It is theorized that such crystallization hampers the ability of the removal composition to wick underneath the nail coating. Stearic acid was found to work synergistically with lauric acid to provide the best results.

TABLE 8

| | | Average removal time |
|---|---|---|
| % wt. Stearic Acid | % wt. Lauric Acid | (seconds) |
| 0 | 0 | 26 |
| 10 | 0 | 25 |
| 20 | 0 | 88 |
| 10 | 10 | 22 |

### Example 11

Various formulations having different levels of methyl laurate were tested. Results are shown in Table 9. Notably, the compositions with methyl laurate failed to outperform the control. It is theorized that removal of the carboxylic acid end group from the carbon backbone has a detrimental effect on removal efficiency, and that the acidic functionality can provide key benefits.

TABLE 9

| | Average removal time |
|---|---|
| % wt. Methyl Laurate | (seconds) |
| 0 | 25 |
| 10 | 33 |
| 20 | 29 |
| 30 | 36 |
| 40 | 39 |

### Example 12

Various formulations having different levels of methyl oleate were tested. One user tested each formulation on 1-2 different fingers. Results are shown in Table 10. Notably, the compositions with methyl oleate failed to outperform the control. It is theorized that removal of the carboxylic acid end group from the carbon backbone has a detrimental effect on removal efficiency, and that the acidic functionality provides key benefits.

TABLE 10

| | Average removal time |
|---|---|
| % wt. Methyl Oleate | (seconds) |
| 0 | 9 |
| 10 | 24 |
| 20 | 12 |
| 30 | 10 |
| 40 | 19 |

### Example 13

Various fatty alcohols were tested. Average removal times at various concentrations of fatty alcohols are shown in Table 11.

TABLE 11

| | 0% | 10% | 20% | 30% | 40% | |
|---|---|---|---|---|---|---|
| Fatty | (sec- | (sec- | (sec- | (sec- | (sec- | Number |
| alcohol | onds) | onds) | onds) | onds) | onds) | of users |
| Lauryl | 23 | 20 | 21 | 31 | 22 | n = 4 |
| Myristic | 24 | 20 | 28 | 33 | 22 | n = 4 |
| Cetyl | 30 | 35 | 37 | 24 | Not tested | n = 4 |
| Behenyl | 13 | 7 | 9 | Not tested | Not tested | n = 1 |

### Example 14

Cottonseed oil and hempseed oil were tested at various concentrations in the solvent base. Results are shown in Tables 12 and 13. For each concentration, the number of users (out of a total of 4) having their best average removal time at that concentration is shown, along with the overall average removal time considering all four users. This is done to highlight good results that may be otherwise masked in the overall average due to user-to-user differences. As shown, the best concentration range for hempseed oil was about 12-20%, particularly about 12%, which showed the lowest overall average. In addition, all four of the users achieved their best average removal times using a composition with an oil concentration of 12%. Similarly, the concentration range for cottonseed oil was about 12-30%, with the users achieving individual best results at the 12-20% range.

TABLE 12
Hempseed Oil

| Hempseed | | Number of |
|---|---|---|
| Oil Conc. | Avg. removal | users with |
| (% wt.) | time (seconds) | best avg. time |
| 0 | 27 | |
| 3 | 33 | |
| 5 | 27 | |
| 10 | 31 | |
| 12 | 16 | 4 |
| 15 | 26 | |
| 20 | 26 | |
| 30 | 25 | |
| 45 | 43 | |
| 60 | 43 | |
| 70 | 39 | |
| 80 | 64 | |
| 90 | 45 | |
| 100 | 71 | |

TABLE 13
Cottonseed Oil

| Cottonseed | | Number of |
|---|---|---|
| Oil Conc. | Avg. removal | users with |
| (% wt.) | time (seconds) | best avg. time |
| 0 | 20 | |
| 3 | 23 | |
| 5 | 17 | |
| 10 | 23 | |
| 12 | 17 | 2 |
| 15 | 16 | |
| 20 | 20 | 2 |
| 30 | 16 | |
| 45 | 25 | |
| 60 | 34 | |
| 70 | 47 | |
| 80 | 53 | |
| 90 | 69 | |
| 100 | 56 | |

It is theorized that hempseed oil performs well as the oil component because of its relatively high linolenic acid content (19-26%).

### Example 15

Three different oil components (cottonseed oil, hempseed oil, and chia seed oil) were tested. Each composition contained 12% oil component in the 99% IPA and methyl acetate (55:45) solvent base. Results are shown in Table 14.

TABLE 14

| | | Number of |
|---|---|---|
| | Avg. removal | users with |
| Type of oil | time (seconds) | best avg. time |
| Chia seed | 22 | 5 |
| Hempseed | 23 | 3 |
| Cotton seed | 22 | 4 |

### Example 16

Five different oil components (cottonseed, wheat germ, perilla seed, pumpkin seed, hempseed) were tested. Each composition contained 12% oil component in the 99% IPA and methyl acetate (55:45) solvent base. Results are shown in Table 15. The tabulated averages are based on a total of 12 removals among 7 users. All tested compositions provided effective and rapid removal without damage to the nail plate, with the wheat germ oil (12%) composition providing the best results.

TABLE 15

| | Avg. removal time |
|---|---|
| Type of oil | (seconds) |
| Cotton seed | 19 |
| Wheat germ | 10 |
| Perilla seed | 13 |
| Pumpkin seed | 14 |
| Hemp seed | 14 |

### Example 17

Nail coating removal compositions were formulated by mixing the components shown in Table 16. These nail coating compositions are readily applied to nail coatings to effect removal of the nail coatings.

TABLE 16

| Composition No. | Component | % weight |
|---|---|---|
| 1 | Solvent | 88 |
| | Cotton seed oil | 7 |
| | Wheat germ oil | 4 |
| | Lauric acid | 1 |
| 2 | Solvent | 88 |
| | Cotton seed oil | 7 |
| | Wheat germ oil | 4 |
| | Lauric acid | 0.5 |
| | Stearic acid | 0.5 |
| 3 | Solvent | 88 |
| | Cotton seed oil | 7 |
| | Wheat germ oil | 4 |
| | Stearic acid | 0.5 |
| | Stearyl alcohol | 0.5 |
| 4 | Solvent | 88 |
| | Cotton seed oil | 7 |
| | Wheat germ oil | 2 |
| | Hemp seed oil | 2 |
| | Lauric acid | 0.5 |
| | Stearic acid | 0.5 |

### Example 18

The oil components for two solvent-based formulations were formed using a 9:3 ratio of cottonseed oil to wheat germ oil. A first formula consisted of the cottonseed and wheat germ oil blend at 12%, lauric acid at 3%, and solvent at 85%. A second formula consisted of the cottonseed and wheat germ oil blend at 12% and the solvent at 88%. Testing on nine users resulted in average removal times of 14 seconds and 12 seconds, respectively.

### Example 19

Various natural oil blends made using a cottonseed oil base were compared against a pure cottonseed oil standard, and average reductions in removal times relative to the cottonseed oil standard were measured. Results are shown in Table 17.

TABLE 17

| | Formulation (in | Avg. reduction in | Number of users |
|---|---|---|---|
| Blend No. | cottonseed oil base) | removal time | (n) |
| 1 | 2% Caprylic acid | 10% | 7 |
| 2 | 4% Myristic acid | 6% | 7 |
| 3 | 2% Myristic acid | 16% | 7 |
| 4 | 0.66% Myristic acid | No reduction | 7 |
| 5 | 2% Myristic alcohol | No reduction | 9 |
| 6 | 2% Cetyl alcohol | No reduction | 9 |
| 7 | 2% Stearyl alcohol | 29% | 9 |
| 8 | 2% Stearic acid | 19% | 9 |
| 9 | 20% Oleic acid | 9% | 6 |
| 10 | 20% Linoleic acid | 4% | 6 |
| 11 | 10% Lauric acid | 30% | 7 |
| 12 | 6% Lauric acid | 16% | 7 |
| 13 | 4% Lauric acid | 13.5% | 7 |
| 14 | 30% Hempseed oil | 20% | 7 |
| 15 | 2% Babussu oil | 14% | 7 |
| 16 | 2% Coconut oil | No reduction | 7 |
| 17 | 2% Lauric acid and | No reduction | 4 |
| | 2% Palmitic acid | | |
| 18 | 4% Palmitic acid | No reduction | 4 |
| 19 | 10% Wheat germ oil | 15% | 7 |
| 20 | 20% Wheat germ oil | 29% | 7 |
| 21 | 30% Wheat germ oil | 11% | 7 |
| 22 | 40% Wheat germ oil | 3% | 7 |
| 23 | 1.5% Behenyl | 13% | 7 |
| | alcohol | | |
| 24 | 1.5% C32-C36 | No reduction | 7 |
| | alcohols | | |

### Example 20

Fatty acid profiles for various natural oil blends listed in Example 17 were calculated using the fatty acid profile information listed in Table 18 (fatty acid amounts of oils listed as % wt.).

TABLE 18

| | Linolenic | Linoleic | Oleic | Stearic | Palmitic | Myristic | Lauric | Capric | Caprylic |
|---|---|---|---|---|---|---|---|---|---|
| Oil | C18:3 | C18:2 | C18:1 | C18 | C16 | C14 | C12 | C10 | C8 |
| Cottonseed | | 53 | 21 | 4.5 | 21.5 | | | | |
| Flaxseed | 35-60 | 17-24 | 12-34 | 2-5 | 4-7 | | | | |
| Grapeseed | 0.1 | 70 | 16 | 4 | 7 | | | | |
| Coconut 91° | | | 5-8 | 1.3 | 7.5-10.5 | 13-19 | 44-52 | 4.5-9.5 | 5.5-9.5 |
| Coconut | | | | | | | | 27.6 | 72.3 |
| (fractionated) | | | | | | | | | |
| Babussu | | | 12-18 | | 6-9 | 15-20 | 44-46 | 2.7-7.6 | 4-6.5 |
| Hempseed | 24-26 | 54-56 | 11-13 | 1-3 | 5-7 | | | | |
| Corn | | 34-62 | 19-49 | 2-5 | 8-12 | 0.2-1 | | 7 | 4 |
| Safflower | | 73-79 | 13-21 | 1-4 | 3-6 | | | | |
| Poppyseed | 5 | 72 | 11 | 2 | 10 | | | | |
| Sunflower | | 44-75 | 14-35 | 1-3 | 3-6 | | | | |
| Walnut | 14 | 58 | 13 | | | | | | |
| Soya bean | 5-11 | 43-56 | 22-34 | 2-6 | 7-11 | | | | |
| Canola | 8.6 | 20 | 63 | 2 | 4 | | | | |
| Chia seed | 62 | 17 | 11 | 2.67 | 6.69 | | | | |
| Perilla | 62-65 | 13-15 | 12-14 | 1-3 | 6-8 | | | | |
| Palm Kernel | | 0.5-2 | 11-19 | 1-3 | 7-9 | 14-18 | 40-52 | 3-7 | 3-5 |
| Palm | | 5-11 | 38-52 | 2-7 | 32-45 | 0.5-2 | | | |
| Olive | | 4-10 | 65-80 | 1-3 | 7-16 | .1-1.2 | | | |
| Wheat germ | 4-10 | 55-60 | 13-21 | 2 | 13-20 | | | | |
| Pumpkin seed | | 64 | 11 | 9.9 | 14 | | | | |
| Camellia | | 7-10 | 80-88 | 1-2 | 7-9 | | | | |
| Evening | 8-14 | 65 | 6-11 | 2 | 6-7 | | | | |
| primrose | | | | | | | | | |
| Rosehip seed | 29-36 | 41-49 | 29-36 | 1-3 | 3-5 | | | | |

The fatty acid distribution (in % wt. of the composition) of different fatty acids and the distribution within various ranges (e.g., C8 to C18) of various removal compositions are shown in Tables 19 and 20, respectively. The data shown in Table 18 can also be used to calculate the fatty acid profiles of other formulations described herein and/or to formulate other natural oil removal compositions based on a desired fatty acid profile.

TABLE 19

| | | | | | C18 | | | | | |
|---|---|---|---|---|---|---|---|---|---|---|
| Blend No. | C18:3 | C18:2 | C18:l | C18:0 | (all) | C16 | C14 | C12 | C10 | C8 |
| 1 | 0 | 52 | 21 | 4 | 77 | 21 | 0 | 0 | 0 | 2 |
| 2 | 0 | 51 | 20 | 4 | 75 | 21 | 4 | 0 | 0 | 0 |
| 3 | 0 | 52 | 21 | 4 | 77 | 21 | 2 | 0 | 0 | 0 |
| 7 | 0 | 52 | 21 | 6 | 79 | 21 | 0 | 0 | 0 | 0 |
| 8 | 0 | 52 | 21 | 6 | 79 | 21 | 0 | 0 | 0 | 0 |
| 9 | 0 | 42 | 37 | 4 | 83 | 17 | 0 | 0 | 0 | 0 |
| 11 | 0 | 48 | 19 | 4 | 71 | 19 | 0 | 10 | 0 | 0 |
| 12 | 0 | 50 | 20 | 4 | 74 | 20 | 0 | 6 | 0 | 0 |
| 13 | 0 | 51 | 20 | 4 | 75 | 21 | 0 | 4 | 0 | 0 |
| 14 | 8 | 54 | 18 | 4 | 83 | 17 | 0 | 0 | 0 | 0 |
| 15 | 0 | 52 | 21 | 4 | 77 | 21 | 0 | 1 | 0 | 0 |
| 19 | 1 | 53 | 21 | 4 | 79 | 21 | 0 | 0 | 0 | 0 |
| 20 | 1 | 54 | 20 | 4 | 80 | 21 | 0 | 0 | 0 | 0 |
| 21 | 2 | 54 | 20 | 4 | 80 | 20 | 0 | 0 | 0 | 0 |
| 22 | 3 | 55 | 19 | 4 | 81 | 20 | 0 | 0 | 0 | 0 |

TABLE 20

| | C8- | C8- | C8- | C8- | C8- | C10- | C10- | C10- |
|---|---|---|---|---|---|---|---|---|
| Blend No. | C10 | C12 | C14 | C16 | C18 | C12 | C14 | C16 |
| 1 | 2 | 2 | 2 | 23 | 100 | 0 | 0 | 21 |
| 2 | 0 | 0 | 4 | 25 | 100 | 0 | 4 | 25 |
| 3 | 0 | 0 | 2 | 23 | 100 | 0 | 2 | 23 |
| 7 | 0 | 0 | 0 | 21 | 100 | 0 | 0 | 21 |
| 8 | 0 | 0 | 0 | 21 | 100 | 0 | 0 | 21 |
| 9 | 0 | 0 | 0 | 17 | 100 | 0 | 0 | 17 |
| 11 | 0 | 10 | 10 | 29 | 100 | 10 | 10 | 29 |
| 12 | 0 | 6 | 6 | 26 | 100 | 6 | 6 | 26 |
| 13 | 0 | 4 | 4 | 25 | 100 | 4 | 4 | 25 |
| 14 | 0 | 0 | 0 | 17 | 100 | 0 | 0 | 17 |
| 15 | 0 | 1 | 1 | 23 | 100 | 1 | 1 | 23 |
| 19 | 0 | 0 | 0 | 21 | 100 | 0 | 0 | 21 |
| 20 | 0 | 0 | 0 | 21 | 100 | 0 | 0 | 21 |
| 21 | 0 | 0 | 0 | 20 | 100 | 0 | 0 | 20 |
| 22 | 0 | 0 | 0 | 20 | 100 | 0 | 0 | 20 |

| Composition | C10- | C12- | C12- | C12- | C14- | C14- | C16- |
|---|---|---|---|---|---|---|---|
| No. | C18 | C14 | C16 | C18 | C16 | C18 | C18 |
| 1 | 98 | 0 | 21 | 98 | 21 | 98 | 98 |
| 2 | 100 | 4 | 25 | 100 | 25 | 100 | 96 |
| 3 | 100 | 2 | 23 | 100 | 23 | 100 | 98 |
| 7 | 100 | 0 | 21 | 100 | 21 | 100 | 100 |
| 8 | 100 | 0 | 21 | 100 | 21 | 100 | 100 |
| 9 | 100 | 0 | 17 | 100 | 17 | 100 | 100 |
| 11 | 100 | 10 | 29 | 100 | 19 | 90 | 90 |
| 12 | 100 | 6 | 26 | 100 | 20 | 94 | 94 |
| 13 | 100 | 4 | 25 | 100 | 21 | 96 | 96 |
| 14 | 100 | 0 | 17 | 100 | 17 | 100 | 100 |
| 15 | 100 | 1 | 22 | 100 | 22 | 99 | 98 |
| 19 | 100 | 0 | 21 | 100 | 21 | 100 | 100 |
| 20 | 100 | 0 | 21 | 100 | 21 | 100 | 100 |
| 21 | 100 | 0 | 20 | 100 | 20 | 100 | 100 |
| 22 | 100 | 0 | 20 | 100 | 20 | 100 | 100 |

### Example 21

Additional nail coating removal compositions were formulated by mixing the components shown in Table 21. The nail coating removal compositions were readily applied to nail coatings to effect removal of the nail coatings.

TABLE 21

| Composition No. | Component | % weight |
|---|---|---|
| 1 | Methyl acetate | 33.7 |
| | IPA 91% | 39.7 |
| | Coconut oil 91° | 15.3 |
| | Flaxseed oil | 8.15 |
| | Tocopherol | 0.09 |
| | Met-10U | 3 |
| 2 | Methyl acetate | 31.5 |
| | IPA 91% | 37 |
| | Cetyl alcohol | 15.6 |
| | Cottonseed oil (RBDW) | 15.9 |
| 3 | Methyl acetate | 35 |
| | IPA 91% | 40.8 |
| | Lauric acid | 2 |
| | Myristic acid | 2 |
| | Cetyl alcohol | 10.1 |
| | Cottonseed oil (RBDW) | 10.1 |
| 4 | Methyl acetate | 33.0 |
| | Cetyl alcohol | 33.0 |
| | Lauric acid | 11.0 |
| | Myristic acid | 11.0 |
| | Caprylic/capric acid | 11.0 |
| | Jojoba oil | 0.95 |
| | Tocopherol | 0.05 |
| 5 | Methyl acetate | 30.0 |
| | IPA 91% | 38.0 |
| | Meadowfoam seed oil | 32.0 |
| 6 | IPA 91% | 56.7 |
| | Palm kernel flake | 24.0 |
| | Coconut oil (fractionated) | 19.3 |
| 7 | Methyl acetate | 33.0 |
| | IPA 91% | 380 |
| | Lauric acid | 29.0 |
| 8 | Methyl Acetate | 32.7 |
| | Isopropyl Alcohol 91% | 38.2 |
| | Coconut Oil 91° | 14.7 |
| | Monolaurin 95% | 14.4 |
| 9 | Methyl Acetate | 27.7 |
| | Isopropyl Alcohol 91% | 37.8 |
| | Flaxseed oil | 12.1 |
| | Monolaurin 95% | 12.1 |
| | Grapeseed oil | 10.3 |

The described embodiments are to be considered in all respects only as illustrative and not restrictive. The scope of the disclosure is, therefore, indicated by the appended claims rather than by the foregoing description. Elements described in relation to any embodiment described herein may be combined with elements described in relation to any other embodiment described herein.

1. A nail coating removal composition, comprising:
a solvent having an alcohol component and an alkyl ester of a carboxylic acid; and
a natural oil blend, wherein the natural oil blend is included in an amount ranging from about 5% to about 30% of the weight of the composition.

2. The composition of claim 1, wherein the alcohol component is isopropyl alcohol.

3. The composition of any one of claim 1, wherein the short chain ester component is methyl acetate.

4. The composition of claim 1, wherein the nail coating composition is formulated to avoid damaging a nail plate to which it is contacted, or is formulated to increase the flexibility, durability, or toughness of the nail plate.

5. The composition of claim 1, wherein the natural oil blend is formulated from plant-based oils.

6. The composition of claim 1, wherein the natural oil blend is formulated from the group consisting of: cottonseed, flaxseed, grapeseed, coconut, avocado, argan, babussu, hempseed, corn, safflower, poppyseed, sunflower, walnut, soya bean, canola, chia seed, perilla seed, palm kernel, peanut, palm, jojoba, macadamia nut, sesame seed, meadowfoam seed, pumpkin seed, wheat germ, and olive oil; caprylic, capric, lauric, myristic, palmitic, stearic, palmitoleic, oleic, linoleic, linolenic, and stearidonic acid, and fatty acid alcohols thereof.

7. The composition of claims 1, wherein an amount of fatty acid alkyl esters included in the composition is less than 5% by weight of the composition.

8. The composition of claim 1, wherein substances insoluble in the alcohol component of the solvent are omitted.

9. The composition of claim 1, wherein urea is omitted.

10. The composition of claim 1, wherein the natural oil blend is included in an amount ranging from about 10% to about 20% of the weight of the composition.

11. The composition of claim 1, wherein the alcohol component makes up about 40 to 70% by weight of the solvent.

12. The composition of claim 1, wherein the ratio of the alcohol component to the short chain ester component is about 4:5 to about 5:4.

13. The composition of claim 1, wherein one or more components of the composition function to disrupt cohesive integrity of a nail coating, and wherein one or more components of the composition function to disrupt an adhesive bond between the nail coating and a nail plate.

14. The composition of claim 1, wherein the natural oil blend includes one or more of cottonseed oil, hempseed oil, wheat germ oil, chia seed oil, flaxseed oil, grapeseed oil, perilla seed oil, or pumpkinseed oil.

15. The composition of claim 1, wherein the natural oil blend has a fatty acid profile of about 90% or more of C16 to C18 fatty acids or fatty alcohols.

16. The composition of claim 1, wherein the natural oil blend has a fatty acid profile of about 65% or more of C18 fatty acids or fatty acid alcohols.

17. A nail coating removal composition, comprising:
a solvent having an isopropyl alcohol component and a methyl acetate component, the isopropyl alcohol to methyl acetate ratio ranging from about 4:5 to about 5:4; and
a natural oil blend having a fatty acid profile of about 65-90% C18 fatty acids.

18. The composition of claim 17, wherein the natural oil blend is included in an amount ranging from about 10% to about 20% of the weight of the composition.

19. A method of removing a nail coating from a nail plate of a wearer, the method comprising:
providing a nail coating removal composition, the nail coating removal composition including:
a solvent having an alcohol component and an alkyl acetate component; and
a natural oil blend, wherein the natural oil blend is included in an amount ranging from about 10% to about 20% of the weight of the composition; contacting the nail coating removal composition to a nail coating; and removing the nail coating from the nail plate.

20. The method of claim 19, wherein the nail coating is a solid nail coating.
